(19) United States
(12) Patent Application Publication
(10) Pub. No.: US 2007/0213656 A1
(43) Pub. Date: Sep. 13, 2007

(54) Device for outputting a qualitative indication associated with the inflation of an expandable member

(76) Inventor: Arthur Ferdinand, San Jose, CA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/369,846

(22) Filed: Mar. 8, 2006

(51) Int. Cl. A61M 31/00

(52) U.S. Cl. 604/65

(57) ABSTRACT

Apparatuses for sensing and outputting data associated with the inflation of an expandable member are described herein. In one embodiment, for example, An apparatus includes a sensor and an output device. The sensor is configured to be coupled to a reservoir that supplies a fluid to an expandable member, and is further configured to output a signal associated with a pressure of the fluid. The output device is configured to be in communication with the sensor. The output device is further configured to output a qualitative pressure indication associated with the signal associated with the pressure of the fluid without outputting a quantitative pressure indication.

### BACKGROUND

The invention relates generally to a medical device, and more particularly to an apparatus for sensing and qualitatively outputting data associated with the inflation of an expandable member when deployed in interior body regions of humans or other animals.

Expandable members are used, for example, to repair fractures or other bone defects. During such procedures, the inflation characteristics of the expandable member can be monitored. For example, the inflation pressure can be monitored to ensure that the balloon or any other portion of the system, including the supply tubes and/or connection devices, does not fail. Similarly, inflation characteristics also can be monitored to ensure that the expandable member does not overexpand, causing damage to the patient.

Known devices for deploying balloons in surgical procedures often provide pressure measurement with output of quantitative pressure data, for example via a pressure gauge. Such devices are often incorporated into the syringe used to supply the pressurized fluid to the balloon. Quantitative measurements, however, are often not desirable because different medical devices may have different system limits, different sizes, etc. Such a system, however, may not be appropriate in all circumstances, such as where pressure information alone is insufficient.

Thus, a need exists for a medical device that provides different forms and/or types of information about the inflation characteristics of an expandable member and/or the system used to deploy the expandable member.

### SUMMARY

Apparatuses for sensing and outputting data associated with the inflation of an expandable member are described herein. In one embodiment, for example, an apparatus includes a sensor and an output device. The sensor is configured to be coupled to a reservoir that supplies a fluid to an expandable member, and is further configured to output a signal associated with a pressure of the fluid. The output device is configured to be in communication with the sensor. The output device is further configured to output a qualitative pressure indication associated with the signal associated with the pressure of the fluid without outputting a quantitative pressure indication.

### BRIEF DESCRIPTION OF THE DRAWINGS

FIG. 1 is a schematic illustrating a sensor and an output device according to an embodiment of the invention.

FIG. 2 is a perspective view of a reservoir and an output device according to an embodiment of the invention.

FIG. 3 is a perspective view of an output device configured to output both a qualitative pressure indication and a qualitative flow indication according to an embodiment of the invention.

FIG. 4 is a perspective view of an output device that is not coupled directly to the reservoir according to an embodiment of the invention.

FIG. 5 is a tabular representation of a series of constant threshold settings according to an embodiment of the invention.

FIG. 6 is a graphical representation of a series of constant threshold settings and an inflation curve for a low-compliant expandable member according to an embodiment of the invention.

FIG. 7 is a graphical representation of a series of constant threshold settings and an inflation curve for a high-compliant expandable member according to an embodiment of the invention.

FIG. 8 is a graphical representation of threshold settings defined as regions that vary with a pressure and a volume according to an embodiment of the invention.

FIG. 9 is a graphical representation of threshold settings that vary with both a pressure and a volume according to an embodiment of the invention.

FIG. 10 is a plan view of a receiver for receiving an identification associated with the medical device according to an embodiment of the invention.

FIG. 11 is a flow chart illustrating a method for transmitting a fluid from a reservoir to an expandable member according to an embodiment of the invention.

### DETAILED DESCRIPTION

In one variation, the apparatus includes a sensor and an output device. The sensor is configured to be coupled to a reservoir that supplies a fluid to an expandable member, and is further configured to output a signal associated with a pressure of the fluid. The output device is configured to be in communication with the sensor. The output device is further configured to output a qualitative pressure indication associated with the signal associated with the pressure of the fluid without outputting a quantitative pressure indication. In another variation, the sensor is configured to output a signal associated with a flow rate and/or volume of the fluid, and the output device is configured to provide a volume indication. In yet another variation, the apparatus is configured to provide both qualitative and quantitative indications associated with a pressure, a volume and/or a flow rate of the fluid.

In some embodiments of the invention, the apparatus is configured to provide two or more qualitative pressure indications. For example, each of the qualitative pressure indications can be associated with a predefined pressure threshold from a set of predefined pressure thresholds. The output device is configured to output the first qualitative pressure indication when the pressure of the fluid crosses the first pressure threshold from the set of predefined pressure thresholds.

In some embodiments of the invention, the sensor is configured to output a signal associated with a flow rate and/or a volume of the fluid, and the output device is configured to output an indication as a function of the signal associated with the flow rate and/or the volume of the fluid. The flow rate and/or the volume of the fluid can be correlated to the volume of the expandable member. Additionally, the output device can be configured to output separate indications associated with two or more parameters (e.g., the pressure, the flow rate and/or the volume of the fluid). Alternatively, the output device is configured to provide a single indication based on two or more parameters (e.g., the pressure, the flow rate and/or the volume of the fluid).

In yet other embodiments of the invention, an apparatus includes a receiver and an output device in communication with the receiver. The receiver is configured to receive an identification associated with a medical device having an expandable member and a catheter configured to communicate a fluid to the expandable member. The output device is configured to output an indication based on the identification.

As used in this specification and the appended claims, the singular forms “a, “a” and “the” include plural referents unless the context clearly dictates otherwise. Thus, for example, the term “a member” is intended to mean a single member or a combination of members, “a material” is intended to mean one or more materials, or a combination thereof.

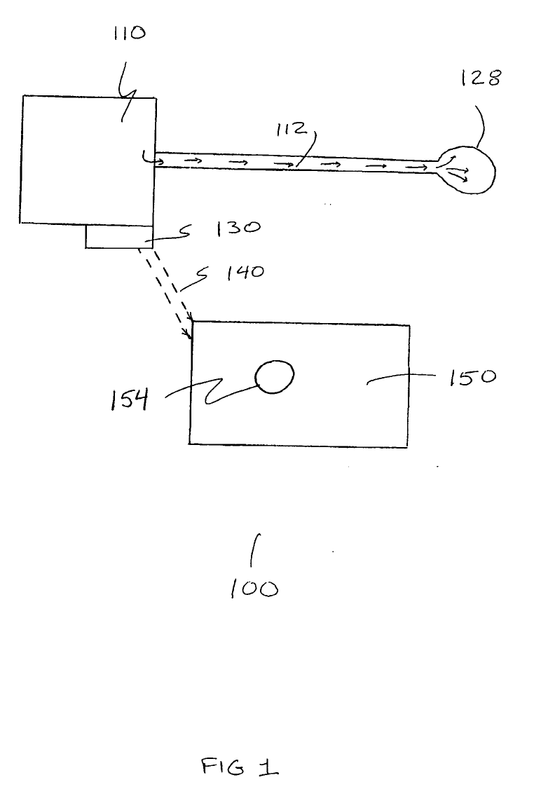

FIG. 1 is a schematic illustration of a medical device 100 according to an embodiment of the invention. The medical device 100 conveys a fluid 112 to an expandable member 128. The medical device 100 includes a reservoir 110 that contains the fluid 112 and a sensor 130 that is coupled to the reservoir 110. The sensor 130 measures a characteristic of the fluid 112 and outputs a signal 140 associated with the measured characteristic. The medical device 100 includes an output device 150 that receives the signal 140 and outputs an indication 154 associated with the signal 140.

The expandable member 128 is configured to move between a collapsed configuration and an expanded configuration in which the expandable member 128 is larger than when in the collapsed configuration. The expandable member 128 can be, for example, a balloon configured to expand between a collapsed configuration and an expanded configuration. The expandable member 128 can be expanded by introducing a fluid 112 into the interior of the expandable member 128. The fluid 112 can be, for example, a gas, a liquid, or any other medium that has fluid-like properties, such as a colloid or a slurry.

One or more sensors 130 can be provided to measure pressure of the fluid 112 at a certain location or locations within the medical device 100. Pressure can be measured, for example, in terms of an absolute pressure, a gauge pressure, a rate of change of pressure, or any combination thereof. For example, in some embodiments, the sensor 130 can measure both a pressure of the fluid 112 within the reservoir 110 and a pressure of the fluid 112 within the expandable member 128. In other embodiments, the sensor 130 can measure a pressure differential between two locations, such as, for example, between an upstream location and a downstream location of a flow orifice (not shown in FIG. 1). In yet other embodiments, the sensor 130 can measure multiple pressures at a single location. For example, the sensor 130 can be configured to measure both the static pressure and the dynamic pressure of the fluid 112 at a location where the fluid 112 exits the reservoir 110.

For embodiments where the sensor 130 measures a pressure of the fluid 112, the sensor 130 can include, for example, a piezoelectric pressure transducer, a diaphragm-type pressure transducer, an electromechanical pressure sensor and/or a silicon pressure sensor. In other embodiments, the sensor 130 can include a mechanical device for measuring a pressure, such as a bourdon tube.

In some embodiments, the sensor 130 can measure a flow of the fluid 112. Flow can be measured in terms of a volumetric flow rate (volume per unit of time), a mass flow rate (mass per unit of time), a total volume expelled from the reservoir 110, a total volume of the expandable member 128, or any combination thereof. The sensor 130 can measure the flow of fluid at a single location or at multiple locations within the medical device 100. For example, the sensor 130 can be configured to measure both the volumetric flow rate of the fluid 112 exiting the reservoir 110 and the total volume of the fluid contained in the expandable member 128.

For embodiments where the sensor 130 measures a flow of the fluid 112, the sensor 130 can include, for example, a flow meter based on pressure measurements, such as an orifice-type flow meter or a venturi-type flow meter. In other embodiments, the sensor 130 can include a flow meter based on the momentum imparted by the fluid onto the flow meter, such as a turbine flow meter or a rotameter. In yet other embodiments, the sensor 130 can include a flow meter designed to measure the flow based on the linear position of a member within the reservoir 110. For example, a position sensor can be coupled to a displacement device (e.g., the plunger in a syringe, the piston in a reservoir, etc.) to detect the volume of fluid being displaced from the reservoir and injected into the expandable member. The sensor 130 transmits a signal 140 to the output device 150, which can be configured to display an indication 154 (e.g., a qualitative indication and/or a quantitative value regarding the volume of fluid injected into the expandable member) in response to the signal 140.

In some embodiments, the signal 140 is an electrical signal associated with a characteristic of the fluid 112 measured by the sensor 130. The signal 140 can be either analog or digital. The signal 140 can be communicated to the output device 150 by a physical connection, such as a conductive wire. In other embodiments, the signal 140 can be communicated to the output device 150 by wireless transmission. In still other embodiments, the signal 140 need not be an electrical signal, but can be a hydraulic, pneumatic or mechanical force that acts upon the output device 150.

In some embodiments, the indication 154 is a qualitative indication, such as a light or series of lights. The indication 154 can be, for example, a series of light emitting diodes (“LED's”), each having a different color. Alternatively, the LED's can vary in brightness or flash in response to the signal 140. In another embodiment, the indication is provided on a liquid crystal display (LCD) showing an icon or other indicia. For example, the LCD may indicate “Under Inflated,” “Target Pressure Reached,” “Over Inflation,” and/or “Danger-Pressure Too High” depending on the pressure, flow rate and/or volume detected by the sensor. In another example, both a qualitative indicator and a quantitative indicator (e.g., the pressure, flow rate and/or volume) are displayed on the LCD. In yet other embodiments, the indication may not be visual in nature. For example, the indication can be an audible alarm or a tactile output, such as a vibration.

In some embodiments, the output device 150 processes the signal 140 prior to selectively outputting an indication 154. For example, the signal 140 can be an analog voltage representing a characteristic of the fluid 112, such as a pressure, a flow rate and/or a volume as discussed above. Upon receiving the signal 140, the output device 150 can digitize the voltage and compare the digitized voltage against a series of predetermined threshold settings. When the digitized voltage crosses one of the threshold settings, the output device 150 outputs an indication 154 associated with the particular threshold settings that has been crossed. For example, a first threshold setting can be associated with an indication 154 in the form of a green LED, while a second threshold setting can be associated with an indication 154 in the form a yellow LED. The threshold settings can be a series of constant values or can change as a function of the signal 140 from the sensor 130, as will be discussed in more detail below.

In other embodiments, the output device 150 processes two or more incoming signals 140 in conjunction with data characterizing the medical device 100 to determine whether an indication should be output. In this manner, the indication 154 is not confined to represent solely a pressure or a flow of the fluid 112, but can indicate a condition related to a combination of pressure and flow that is unique to the particular design of the medical device 100. For example, the output device 150 can calculate the pressure drop of the fluid 112 through the medical device 100 based on a pressure measurement, a flow rate measurement, and the physical characteristics of the medical device 100.

The output device 150 can include a processor configured to process the signal 140, as described above. The processor can be a commercially-available processing device dedicated to performing one or more specific tasks. For example, the processor can be a commercially-available microprocessor. Alternatively, the processor can be an application-specific integrated circuit (ASIC) or a combination of ASICs, which are designed to perform one or more specific functions. In yet other embodiments, the processor can be an analog or digital circuit, or a combination of multiple circuits. Similarly, the output device 150 can include a memory device (not shown) configured to receive and store information, such as a series of threshold settings, a digitized signal, and the like. The memory device can include one or more types of memory. For example, the memory device can include a read only memory (ROM) component and a random access memory (RAM) component. The memory device can also include other types of memory suitable for storing data in a form retrievable by a processor, for example, electronically-programmable read only memory (EPROM), erasable electronically-programmable read only memory (EEPROM), or flash memory.

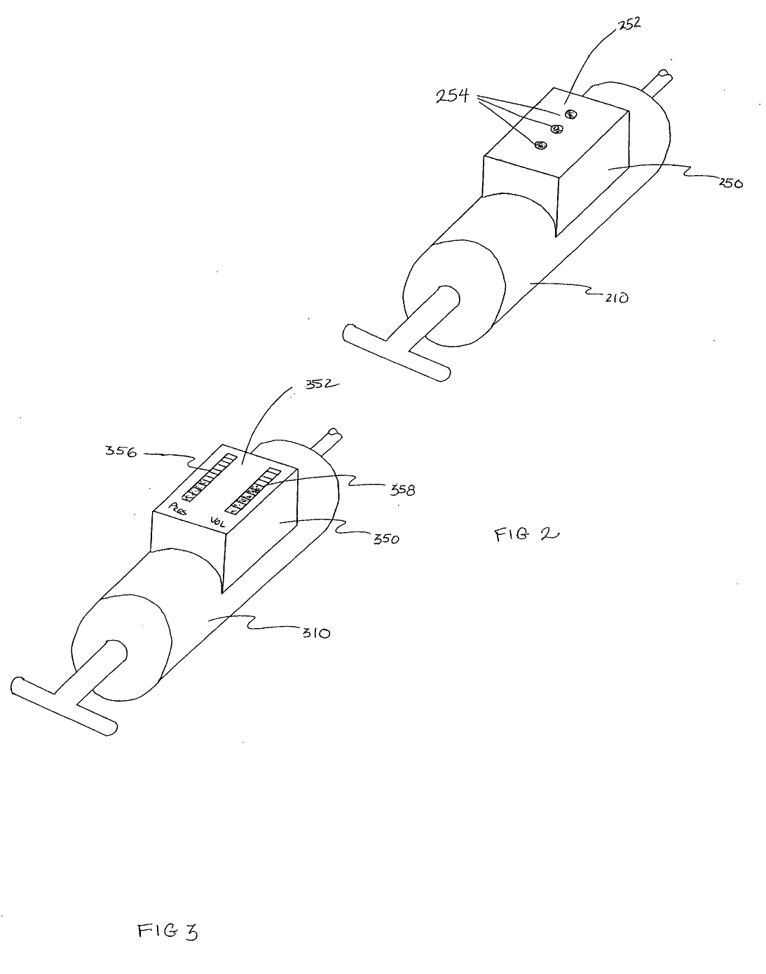

FIG. 2 shows an embodiment of the invention in which the reservoir 210 is a manually-actuated syringe configured to pump a fluid into the expandable member. In other embodiments, the reservoir can be an automatically-actuated syringe. In yet other embodiments the reservoir does not include any mechanism for pressurizing the fluid. For example, the reservoir can be a container filled with a pressurized fluid and having a valve to control the flow of the fluid into the expandable member.

In the embodiment illustrated in FIG. 2, the output device 250 is coupled directly to the reservoir 210, and the sensor (not shown) is fixedly coupled to the output device 250. In this manner, the output device 250 and the sensor can be easily coupled to the reservoir 210 as a single unit. In other embodiments, the sensor can be coupled to the reservoir 210 apart from the output device 250.

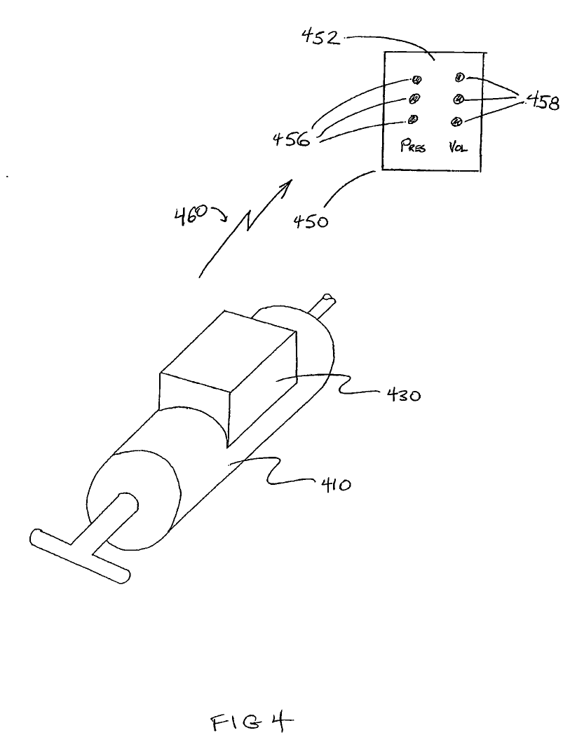

As illustrated in FIG. 2, the indication 254 is a qualitative indication in the form of an LED display. As previously discussed, the indication 254 can be associated with the pressure of the fluid, the flow of the fluid, or an additional parameter calculated by the output device 250. In some embodiments, the output device can include separate indications associated with different parameters. For example, the embodiment illustrated in FIG. 3 includes both a qualitative pressure indication 356 and a qualitative flow indication 358. In this manner, a user can independently monitor the pressure of a fluid and the flow of a fluid.

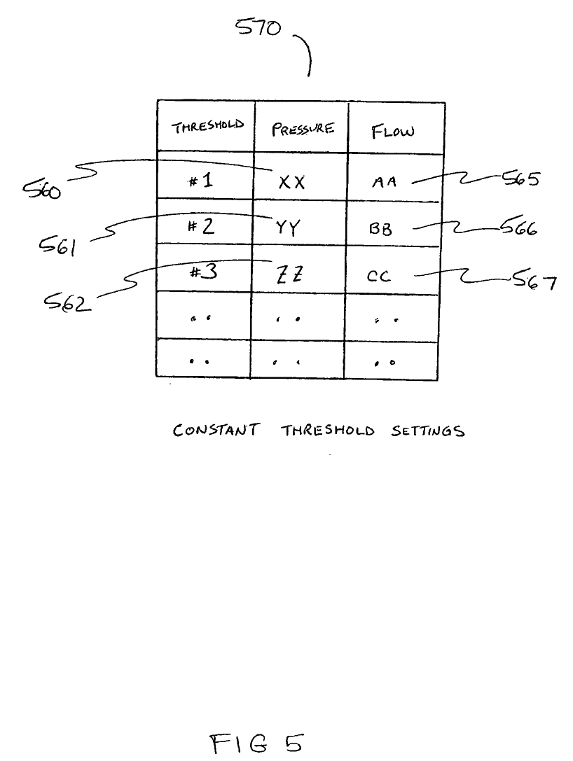

FIG. 4 is a perspective view of an embodiment of the invention in which the output device 450 is not physically coupled to the reservoir 410 or the sensor 430. In the illustrated embodiment, the sensor 430 transmits the signal to the output device 450 by a wireless transmission 460. This configuration allows the user to mount the output device 450 in a location convenient for monitoring. For example, the user can mount the output device 450 adjacent to an imaging system used for one or more aspects of a medical procedure. Such an imaging system could be, for example, a fluoroscopic imaging system used to visualize inflation characteristics of an expandable member during a medical procedure. In such an arrangement, the user can more easily rely on the imaging system to track the intermediate changes in the size and shape of the expandable member, while relying on the output device 450 to provide qualitative indications 456 and 458 only when certain limitations has been reached. This arrangement also allows the user to monitor multiple aspects of the medical procedure without continually shifting focus between the reservoir 410 and the imaging system.

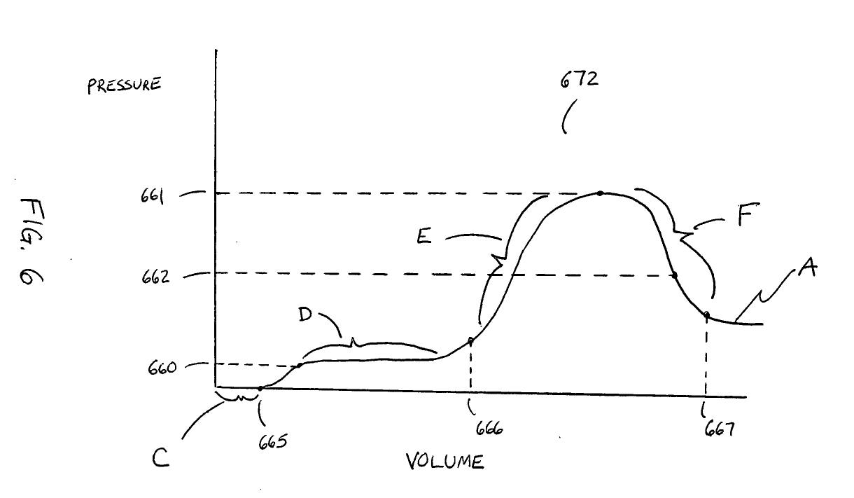

FIG. 5 provides a tabular representation of a series of threshold settings used in some embodiments of the invention. The table of threshold settings 570 includes a series of constant pressure threshold settings 560, 561, and 562 and constant flow threshold settings 565, 566, and 567 that can be stored in a memory device included within an output device. In some embodiments, the output device can contain multiple threshold tables 570, each being uniquely associated with a given medical device. For example, in some embodiments, the output device can contain a first threshold table having values appropriate for and to be used with a medical device having an expandable member having a maximum volume of three cubic centimeters. The output device can also contain a second threshold table having values appropriate for and to be used with a second medical device having an expandable member having a maximum volume of five cubic centimeters. In this manner, a single output device can be used in conjunction with any number of different medical devices.

The first column, labeled as “threshold” includes a representation of whether the threshold setting is a first threshold setting, a second threshold, and so on. The representation can be configured to be associated with an indication, such that when a given threshold setting is crossed, the associated indication is output by the output device. For example, in some embodiments, the first threshold setting can be associated with a green LED, the second threshold setting can be associated with a yellow LED, and so on. A threshold setting may be “crossed” either in an upward or downward direction, as appropriate. For example, as pressure of the fluid increases from below the first threshold to above the first threshold, the first indication is activated.

The second column, labeled as “pressure” includes a series of constant pressure threshold settings 560, 561, and 562. In the illustrated embodiment, the threshold settings represented as XX, YY and ZZ can be in a variety of different formats or units of measure. For example, the first threshold setting 560 can be a static pressure value in a desired units of measurement, such as pounds-per-square-inch. In other embodiments, the first threshold setting 560 can be in the form of a machine-readable code, such as binary or hexadecimal that represents a pressure value. Similarly, the second column, labeled as “flow” includes a series of flow threshold settings 565, 566, and 567.

Although threshold table 570 is shown with only three columns, in other embodiments, it can include any number of columns and rows. For example, in some embodiments the threshold table can include multiple columns of pressure threshold settings. In other embodiments, the threshold table can include a column of threshold settings associated with a calculated parameter that is neither pressure nor flow. Also, while the illustrated example lists only three threshold settings (i.e., three rows) for a given physical characteristic, such as pressure or flow, the threshold table 570 can include any number of threshold settings.

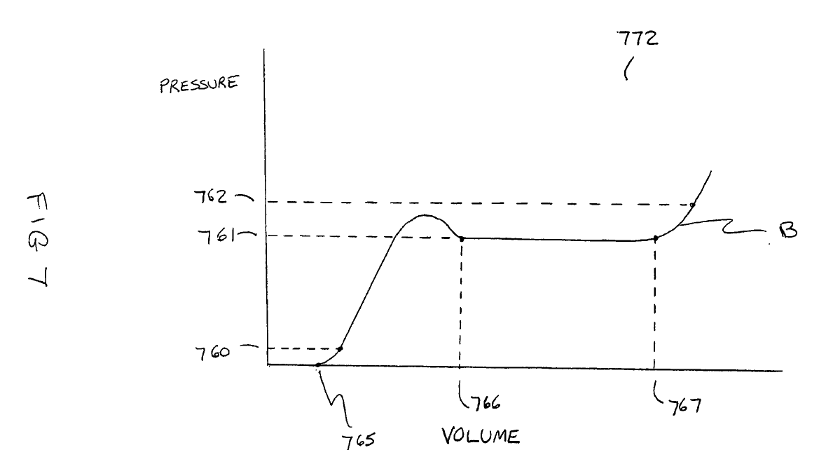

FIG. 6 illustrates a graphical representation 672 of a series of constant pressure and constant flow threshold settings. In the illustrated embodiment, the horizontal axis, labeled as “volume,” corresponds to the volume of an expandable member during inflation. The vertical axis, labeled as “pressure,” corresponds to the pressure within the expandable member during inflation. A solid line represents an example of an inflation curve A for a low-compliant expandable member. A series of dashed lines represents a series of constant pressure threshold settings 660, 661, and 662 and a series of constant flow threshold settings 665, 666, and 667. By representing the threshold settings in a graphical format, the threshold settings can be associated with various regions of the inflation curve A for the expandable member. For example, FIG. 6 shows an initial period of system “take-up” (region C), which can be described as a lag occurring after flow has commenced but before the internal pressure of the expandable member has increased above a nominal value. In the illustrated embodiment, both the first flow threshold setting 665 and the first pressure threshold setting 660 are set to correspond to points at which system “take-up” is complete. In this manner, an output device can output a first indication, such as a green LED, to indicate that system “take-up” is complete.

Once system “take-up” is complete, the internal pressure of a low-compliant expandable member increases somewhat as the expandable member unfolds (region D). After the expandable member is substantially unfolded and/or becomes constrained by hard bone, as more flow is introduced, the internal pressure will increase rapidly (region E) until the expandable member causes the bone to be displaced (region F, discussed in more detail below) and/or the expandable member reaches a substantially expanded configuration. In the illustrated embodiment, the second flow threshold setting 666 corresponds to a transition point between the region of nearly constant pressure (region D) and the region of rapid pressure increase (region E). The second pressure threshold setting 661 corresponds to a point nearing the maximum pressure of the expandable member. In this manner, the second pressure threshold setting 661 is de-coupled from the second flow threshold setting 666, in that they do not correspond to the same point on the inflation curve A. Similarly, the third flow threshold setting 667 corresponds to a point nearing the maximum volume of the expandable member. Finally, the third pressure threshold 662 corresponds to a point along the curve (region F) where the decrease in the pressure of the expandable member indicates that movement of the bone has taken place. In other words, an indication associated with the third pressure threshold 662 is output when the third pressure threshold 662 is crossed from above the threshold to below the threshold (as occurs in region F), but not when the threshold is crossed from below the threshold to above the threshold (as occurs in region E).

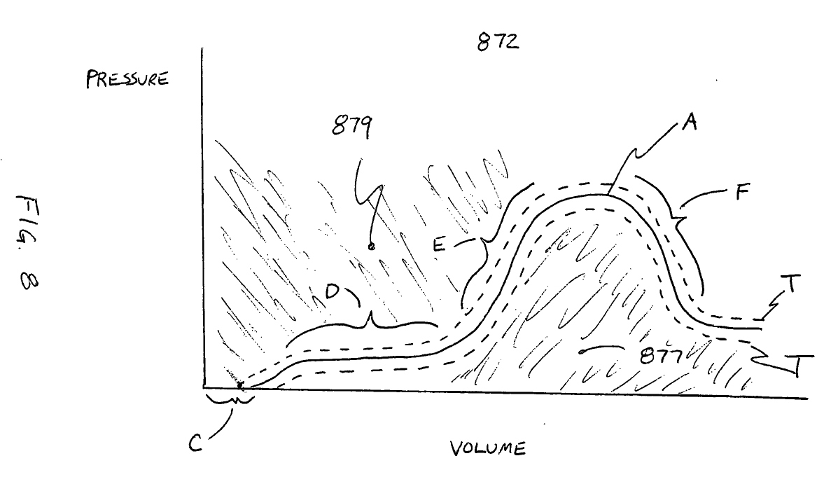

Although the illustrated embodiment includes three pressure threshold settings and three flow threshold settings corresponding to points along an example of an inflation curve for a low-compliant balloon, a graphical representation of threshold settings can include any number of variables and threshold settings. In yet other embodiments, a series of pressure and flow threshold settings can be represented along with an inflation curve having different characteristics. FIG. 7, for example, illustrates a graphical representation 772 of an example inflation curve B for a high-compliance expandable member. These different types of representations can be stored or represented in memory in a number of possible ways, such as in tabular form or in the form of one or more equations from which inflation curves and threshold settings can be calculated.

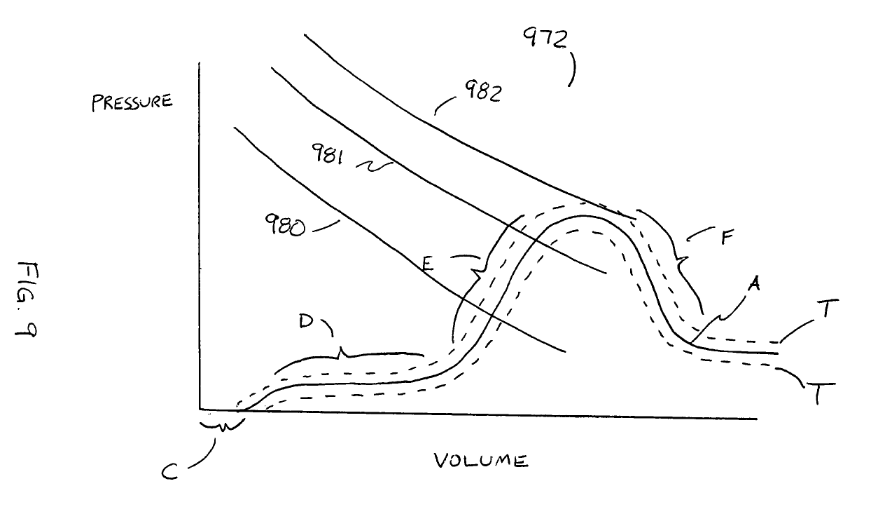

Although the embodiments illustrated in FIG. 5 through FIG. 7 depict threshold settings that are predefined for a given medical device, other embodiments include threshold settings that vary depending on the conditions in which the apparatus is used. For example, FIG. 8 illustrates a graphical representation 872 of an embodiment having threshold settings defined as regions that vary with a pressure and/or a flow. In the illustrated embodiment, the horizontal axis, labeled as “volume,” corresponds to the volume of an expandable member during inflation. The vertical axis, labeled as “pressure,” corresponds to the pressure within the expandable member during inflation. A solid line represents an inflation curve A for a nominal low-compliant expandable member. The two dashed lines represent the system tolerance T associated with the expandable member and the medical device. For example, the maximum volume of an expandable member may vary from 4.8 cubic centimeters to 5.2 cubic centimeters due to normal variance in manufacturing processes. This variance, while expected, may result in a slight change in the inflation characteristics of the expandable member, which are graphically illustrated by the system tolerance T. Although depicted as symmetrical about the nominal inflation curve A, in some embodiments the system tolerance T can be asymmetrical.

The embodiment illustrated in FIG. 8 includes threshold settings 877 and 879 that are defined by the shaded regions. In this manner, the threshold settings are not a series of predefined values, but are instead regions defined as a function of the pressure and flow, bounded by the system tolerance T of the inflation curve A. Threshold region 877 corresponds to inflation conditions during which the internal pressure of the expandable member is lower than would be expected based on the nominal inflation curve A and system tolerance T. For example, if the system experiences a leak, such as a ruptured expandable member, the relationship between the flow and pressure will not correspond to the inflation curve A of the expandable member. In such cases, while neither the pressure or flow alone may be sufficient to cross a constant threshold settings, the combination of pressure and flow crosses into threshold region 877, thereby triggering the output device to output an indication associated with region 877.

Conversely, threshold region 879 corresponds to inflation conditions during which the internal pressure of the expandable member is higher than would be expected based on the nominal inflation curve A and system tolerance T. For example, if the expandable member becomes externally restricted by a structure within a patient, such as a bone, it may not unfold in a manner consistent with the nominal inflation curve A. This condition can result in higher pressure within the expandable member than would be seen in cases where the expandable member is inflated without external restriction. In such cases, the combination of pressure and flow crosses into threshold region 879, thereby triggering the output device to output an indication associated with region 879. The indication can be, for example, a warning to the user that the expandable member is in position to displace a portion of a fractured vertebrae.

Although FIG. 8 only illustrates two threshold regions based on flow and pressure, other embodiments can include any number of threshold regions based on any number of measured or calculated inflation characteristics. In some embodiments, the threshold settings are defined as regions defined by the temporal change in a pressure and/or a flow. For example, when an expandable member is held at a certain level of inflation, a subsequent slow decay in pressure can indicate that a portion of a fractured vertebrae is being displaced. A subsequent rapid decay in pressure, however, can indicate that the expandable member has ruptured. As such, the threshold regions are defined to trigger the output device to output an indication associated with a system failure when a rapid drop in pressure is sensed, but not necessarily output an indication associated with bone displacement when a slow drop in pressure is sensed.

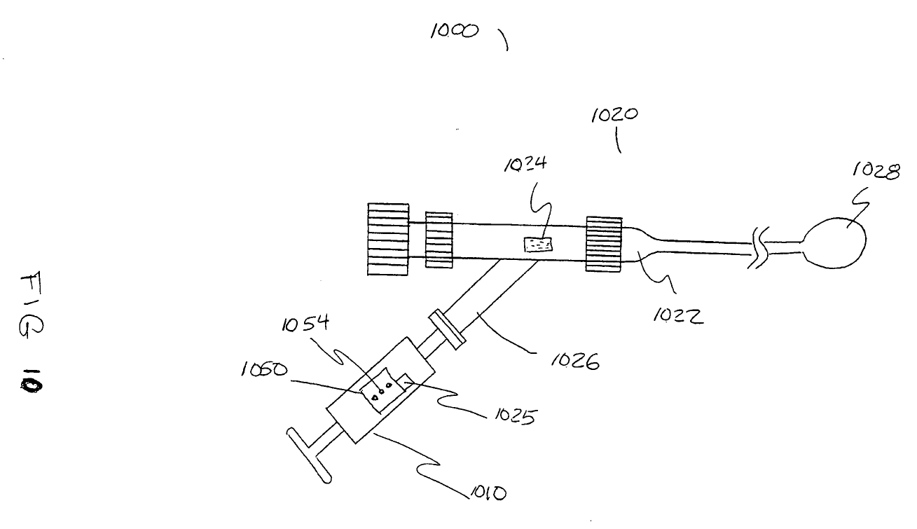

FIG. 9 illustrates a graphical representation 972 of another embodiment having threshold settings that vary as a function of the inflation characteristics of the expandable member. In the illustrated embodiment, the horizontal axis, labeled as “volume,” corresponds to the volume of an expandable member during inflation. The vertical axis, labeled as “pressure,” corresponds to the pressure within the expandable member during inflation. A solid line represents an inflation curve A for a nominal low-compliant expandable member. The two dashed lines represent the system tolerance T associated with the expandable member and the medical device. Three solid lines represent a series of variable pressure threshold settings 980, 981 and 982 that vary as a function of the flow. In this manner, the threshold settings change in response to the changing conditions experienced during inflation.

For example, when an expandable member becomes externally restricted, it may be able to withstand higher internal pressures than when it is inflated in an unconstrained condition. As such, a pressure threshold that is appropriate for an expandable member that is fully expanded may not be appropriate for an expandable member that is partially expanded. The illustrated embodiment accounts for this by allowing the pressure threshold settings 980, 981, and 982 to change as a function of the flow. Although shown as continuous curves, the pressure threshold settings can also vary in a discontinuous manner. For example, in certain conditions, the pressure threshold settings can correspond to the physical limitations of the expandable member, whereas in other conditions, the pressure threshold settings can correspond to the physical limitations of a system component other than the expandable member, such as a supply tube or fitting.

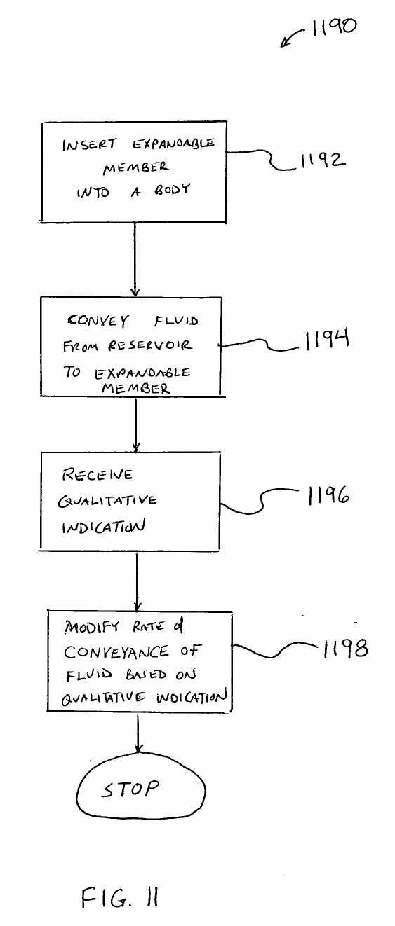

FIG. 10 illustrates an embodiment of the invention that includes a receiver 1025, an output device 1050 in communication with the receiver 1025, and an identifier 1024. The identifier 1024 is configured to provide an identification associated with the medical device 1020. The identification can be, for example, a model number or a serial number characterizing the medical device 1020. In other embodiments, the identification can be data that characterizes the medical device 1020, such as the maximum volume of the expandable member 1028 or a length and a diameter of the catheter 1022 that supplies a fluid to the expandable member 1028. Alternatively, the identification can be data that indicates the thresholds and threshold values.

The identifier 1024 can be a physical tag coupled to the medical device 1020. In some embodiments, the identifier can be a bar code readable tag mounted to the catheter 1022. In other embodiments the identifier can be a radio frequency identification (RFID) tag coupled to the expandable member 1028. In yet other embodiments, the identifier can be a series of conductive pins configured to mate in an identifying manner when physically connected to the receiver 1025. For example, the receiver 1025 can be designed to contact the identifier 1024 in a keyed fashion when the reservoir 1010 is coupled to the inflation port 1026 of the medical device 1020.

The receiver 1025 can receive the identification supplied by the identifier 1024 and communicate the identification to the output device 1050. Although the illustrated embodiment shows the receiver 1025 being adjacent to the output device, in some embodiments the receiver 1025 is not physically connected to the output device.

In the illustrated embodiment, the output device 1050 outputs an indication 1054 based on the identification. The indication can be, for example, a character string representing the serial number of the medical device 1020. In some embodiments, the indication can be a value representing the maximum volume of the expandable member 1028. In other embodiments, the indication can be a pressure indication or a flow indication of the types discussed above that is output when the pressure or flow crosses a predetermined threshold setting. The threshold setting can be determined by the output device 1050 based on the identification. For example, if the identification includes the maximum volume of the expandable member 1028, the output device 1050 can be configured to select an appropriate table of thresholds based on the maximum volume included within the identification. In this manner, the output device automatically selects the appropriate threshold settings, thereby minimizing the opportunity for human error in selecting thresholds. Alternatively, the identification can be the thresholds and threshold values.

FIG. 11 is a flow chart illustrating a method 1190 for conveying a fluid from a reservoir to an expandable member according to an embodiment of the invention. The illustrated method includes inserting (at 1192) an expandable member into the body of a patient. The expandable member can be a low-compliant expandable member of the type discussed above. In some embodiments, the expandable member is inserted percutaneously into an interior region of a vertebra.

At 1194, a fluid is conveyed from a reservoir to the expandable member. As discussed above, the reservoir includes a sensor configured to output a signal associated with a pressure of the fluid. In some embodiments, the reservoir is a syringe that includes a manually-actuated plunger configured to pump the fluid from the syringe into the expandable member. In other embodiments, the reservoir includes an automatically-actuated syringe. In yet other embodiments the reservoir does not include any mechanism for pressurizing the fluid. For example, the reservoir can be a container filled with a pressurized fluid, which is conveyed to the expandable member by adjusting the position of a valve disposed between the reservoir and the expandable member.

At 1196, a qualitative indication associated with the signal associated with the pressure of the fluid is received. As discussed above, the qualitative indication can be received in a variety of different forms, such as visual, audible and/or tactile. In some embodiments, the qualitative indication is received from an output device of the type discussed above. In other embodiments, a method includes receiving a plurality of qualitative indications, each being associated with a different pressure threshold from a plurality of pressure thresholds.

At 1198, the rate of conveyance of the fluid from the reservoir to the expandable member is modified based on the qualitative indication received. In some embodiments, the rate of conveyance is modified by reducing the rate of transmission of the fluid from the reservoir to the expandable member. This can be accomplished in some embodiments, for example, by manually reducing the rate at which the syringe plunger is depressed. In other embodiments, the rate of conveyance is modified by increasing the rate of transmission of the fluid from the reservoir to the expandable member, for example, by manually increasing the rate at which the syringe plunger is depressed. In yet other embodiments, the rate of conveyance is modified by stopping the transmission of the fluid from the reservoir to the expandable member, for example, by closing a valve disposed between the reservoir and the expandable member. In still other embodiments, the rate of conveyance is modified by withdrawing the fluid from the expandable member.

While various embodiments of the invention have been described above, it should be understood that they have been presented by way of example only, and not limitation. Where methods and steps described above indicate certain events occurring in certain order, the ordering of certain steps may be modified. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, although the qualitative indication has been described as a light or a series of lights, one of ordinary skill in the art having the benefit of this disclosure would appreciate that other types of qualitative indications can be used. In one variation, for example, the qualitative indication can be a qualitative gauge that includes a needle configured to point to different regions on the face of the gauge in response to a signal. In another variation, the qualitative indication can be a LCD capable of displaying an indicia that indicates the status of a particular parameter (e.g., pressure, flow rate and/or volume) being monitored by the apparatus.

Although the threshold settings have been described as being one or more predefined, constant values, in some embodiments, the threshold settings vary depending on the particular parameter (e.g., pressure, flow rate and/or volume) being monitored by the apparatus. Similarly, although the graphical representations are shown and described as two-dimensional plots, the graphical representations can be of any type that represents the threshold settings, such as a three-dimensional graph.

What is claimed is:

1. An apparatus, comprising:
a sensor in fluid communication with a reservoir, the reservoir being configured to supply a fluid to an expandable member, the sensor configured to output a signal associated with a pressure of the fluid; and
an output device in communication with the sensor, the output device configured to output a qualitative pressure indication in response to the signal without outputting a quantitative pressure indication.

2. The apparatus of claim 1, further comprising the expandable member, the pressure being at a location within the expandable member.

3. The apparatus of claim 1, wherein:
the output device is coupled to the reservoir; and
the sensor is fixedly coupled to the output device.

4. The apparatus of claim 1, wherein the output device is configured to output the qualitative pressure indication when the pressure of the fluid crosses a pressure threshold.

5. The apparatus of claim 1, wherein:
the qualitative pressure indication is a first qualitative pressure indication from a plurality of qualitative pressure indications, each qualitative pressure indication being associated with a pressure threshold from a plurality of pressure thresholds; and
the output device is configured to output the first qualitative pressure indication when the pressure of the fluid crosses the pressure threshold from the plurality of pressure thresholds.

6. The apparatus of claim 1, wherein the output device includes a visual display configured to display the qualitative pressure indication.

7. The apparatus of claim 1, wherein the output device includes an audible alarm configured to output the qualitative pressure indication.

8. The apparatus of claim 1, wherein the output device is configured to transmit the qualitative pressure indication as a tactile output.

9. An apparatus, comprising:
a sensor in fluid communication with a reservoir that supplies a fluid to an expandable member, the sensor configured to output a signal associated with at least one of a flow rate or a volume of the fluid; and
an output device in communication with the sensor, the output device configured to output at least one indication as a function the signal associated with the at least one of the flow rate or the volume of the fluid.

10. The apparatus of claim 9, wherein the indication is solely qualitative.

11. The apparatus of claim 9, wherein the volume of the fluid corresponds to a volume of the expandable member.

12. The apparatus of claim 9, wherein the output device is configured to output the indication when the at least one of the flow rate or the volume of the fluid crosses a threshold.

13. The apparatus of claim 9, wherein:
the indication is a first indication from a plurality of indications, each indication being associated with a threshold from a plurality of thresholds; and
the output device is configured to output the first indication when the at least one of the flow rate or the volume of the fluid crosses the threshold from the plurality of thresholds.

14. An apparatus, comprising:
a sensor coupled to a reservoir that supplies a fluid to an expandable member, the sensor configured to output a signal associated with a pressure of the fluid and a signal associated with at least one of a flow rate or a volume of the fluid; and
an output device in communication with the sensor, the output device configured to output a pressure indication as a function of the signal associated with the pressure of the fluid and a volume indication as a function the signal associated with the at least one of the flow rate or the volume of the fluid.

15. The apparatus of claim 14, wherein the pressure indication includes a qualitative indication.

16. The apparatus of claim 14, wherein the flow indication includes a qualitative indication.

17. The apparatus of claim 14, wherein the output device is configured to output the pressure indication when the pressure of the fluid crosses a pressure threshold.

18. The apparatus of claim 14, wherein:
the pressure indication is a first pressure indication from a plurality of pressure indications, each pressure indication being associated with a pressure threshold from a plurality of pressure thresholds;
the volume indication is a first volume indication from a plurality of volume indications, each volume indication being associated with a volume threshold from a plurality of volume thresholds;
the output device is configured to output the first pressure indication when the pressure of the fluid crosses the pressure threshold from the plurality of pressure thresholds; and
the output device is configured to output the first volume indication when the at least one of the flow rate or the volume of the fluid crosses the volume threshold from the plurality of volume thresholds.

19. The apparatus of claim 14, wherein the sensor includes:
a pressure sensor configured to output a signal associated with a pressure of the fluid; and
a flow meter configured to output a signal associated with the at least one of the flow rate or the volume of the fluid.

20. The apparatus of claim 14, wherein:
the output device is configured to output the volume indication when the at least one of the flow rate or the volume of the fluid crosses a volume threshold; and
the volume threshold is associated with a volume of the expandable member.

21. An apparatus, comprising:
a sensor coupled to a reservoir that supplies a fluid to an expandable member, the sensor configured to output a signal associated with a pressure of the fluid and a signal associated with at least one of a flow rate or a volume of the fluid; and
an output device in communication with the sensor, the output device configured to output an indication as a function of the signal associated with the pressure and the signal associated with the at least one of the flow rate or the volume.

22. The apparatus of claim 21, wherein:
the output device is configured to output the indication when the pressure of the fluid crosses a pressure threshold; and
the pressure threshold is a function of the at least one of the flow rate or the volume of the fluid.

23. The apparatus of claim 21, wherein:
the indication is a first indication from a plurality of indications, each indication being associated with a pressure threshold from a plurality of pressure thresholds;
the pressure threshold is a function of the at least one of the flow rate or the volume of the fluid; and
the output device is configured to output the first indication when the pressure of the fluid crosses the pressure threshold from the plurality of pressure thresholds.

24. An apparatus, comprising:
a receiver configured to receive an identification associated with a medical device having an expandable member and a catheter configured to convey a fluid to the expandable member; and
an output device in communication with the receiver, the output device configured to output an indication based on the identification.

25. The apparatus of claim 24 wherein the identification is a datum associated with the medical device.

26. The apparatus of claim 24, further comprising an identifier associated with the medical device, the identifier configured to provide the identification associated with the medical device.

27. The apparatus of claim 26 wherein the identifier is associated with a pressure of the fluid being used to expand the expandable member.

28. The apparatus of claim 24, further comprising an identifier associated with the medical device, the identifier configured to provide the identification associated with the medical device, wherein the identifier is coupled to at least one of the catheter or the expandable member.

29. The apparatus of claim 24, further comprising an identifier associated with the medical device, the identifier configured to provide the identification associated with the medical device, and wherein the receiver is configured to be removably and matingly coupled to the identifier.

30. The apparatus of claim 24, wherein:
the indication is a pressure indication associated with a pressure of the fluid; and
the output device is configured to output the pressure indication when the pressure of the fluid crosses a pressure threshold, the pressure threshold being a function of the identification.

31. The apparatus of claim 24, wherein:
the indication is an indication associated with at least one of the flow rate or the volume of the fluid; and
the output device is configured to output the indication when the at least one of the flow rate or the volume of the fluid crosses a threshold, the threshold being a function of the identification.

32. The apparatus of claim 24, wherein:
the indication is qualitative; and
the output device is configured to output the indication without outputting a quantitative pressure indication.

33. A kit, comprising:
an expandable member configured to move between a collapsed configuration and an expanded configuration;
a reservoir configured to convey a fluid to the expandable member, the reservoir including a sensor configured to output a signal associated with a pressure of the fluid within the reservoir; and
an output device in communication with the sensor, the output device configured to output a qualitative pressure indication in response to the signal without outputting a quantitative pressure indication.

34. The kit of claim 33, wherein the output device is coupled to the reservoir.

35. A method, comprising:
inserting an expandable member into a body;
conveying a fluid from a reservoir to the expandable member, the reservoir including a sensor configured to output a signal associated with a pressure of the fluid within the reservoir;
receiving a qualitative indication associated with the signal without receiving a quantitative indication; and
modifying the amount of fluid being conveyed from the reservoir to the expandable member based on the qualitative indication.

36. The method of claim 35, wherein the modifying includes at least one act selected from a group consisting of (1) reducing the rate of conveyance of the fluid from the reservoir to the expandable member, (2) increasing the rate of conveyance of the fluid from the reservoir to the expandable member, (3) stopping the conveyance of the fluid from the reservoir to the expandable member or (4) withdrawing the fluid from the expandable member.

37. The method of claim 35, wherein the expandable member is inserted percutaneously into an interior region of a vertebra.
